(19) United States
(12) Patent Application Publication
(10) Pub. No.: US 2005/0070933 A1
(43) Pub. Date: Mar. 31, 2005

(54) Apparatus and method for intestinal irrigation

(76) Inventor: Arnold R. Leiboff, Stony Brook, NY (US)

Correspondence Address: STEINBERG & RASKIN, P.C., 1140 AVENUE OF THE AMERICAS, 15th FLOOR, NEW YORK, NY, 10036-5803, US

(21) Appl. No.: 10/672,053

(22) Filed: Sep. 26, 2003

(51) Int. Cl. A61B 17/08

(52) U.S. Cl. 606/153; 604/284

(57) ABSTRACT

Drain tube for colonic irrigation including a rigid body having a tubular portion and a side arm extending from a side of the tubular portion. The tubular portion has a proximal end adapted to be inserted into a bowel and a distal end through which an irrigation tube is inserted. The body is formed to be rigid and transparent via an injection molding process. The transparency of the body enables the effluent through the side arm to be visualized so that the colonic irrigation can be stopped when the effluent is clear. Additional improvements to a drain tube and method for using the same are also disclosed.

### FIELD OF THE INVENTION

The present invention relates generally to apparatus and methods for intestinal irrigation and more specifically to improvements to apparatus and methods for intestinal irrigation of the type disclosed in U.S. Pat. No. 4,637,814 (Leiboff).

### BACKGROUND OF THE INVENTION

The importance of a clean colon to the success of colonic surgery is well established. In U.S. Pat. No. 4,637,814, incorporated by reference herein, the inventor presented several innovative apparatus and methods for irrigating and cleaning a colon in preparation for subsequent colonic surgery. One apparatus described in this patent includes a drain tube having a tube body and a single side arm. In use, a proximal end of the tube body is inserted into the colon and an irrigation tube is inserted into a distal end of the tube body and pushed forward to extend beyond the proximal end of the tube body and into the colon. Irrigating fluid is directed through the irrigation tube to clean fecal matter from the colon. Effluent generated during the colonic irrigation procedure flows into the tube body and the side arm and is removed through a tube connected to the side arm. Additional details about this apparatus, related apparatus and methods for using the same can be found in the '814 patent.

A similar drain tube having a tube body and a single side arm is described in U.S. Patent Application Publication No. 2002/0188244. The drain tube described in this patent application publication has a port at the distal end of the tube body which incorporates a sealing mechanism designed to prevent leakage of effluent from the tube body.

The drain tubes in both of these references have drawbacks which detract from their use for an ideal colonic irrigation. For example, the drain tubes are made from a molding process referred to as “dip molding” which results in a soft, pliable drain tube but which also inherently precludes the drain tubes from being sufficiently transparent to enable the color and quality of the effluent to be seen. The colonic irrigation may be stopped based on analysis of the color and quality of the effluent and therefore the inability to clearly view the color and quality of the effluent prevents a precise determination as to when it is preferable to stop the colonic irrigation. This may result in premature cessation of the colonic irrigation, i.e., when the colon is insufficiently cleaned, or a prolonged colonic irrigation in which valuable surgical time is squandered.

It would therefore be desirable to fabricate drain tubes for use in colonic irrigation which are highly transparent and therefore enable an accurate determination of the color and quality of the effluent leading to a precise determination of when the colonic irrigation can be stopped.

There are other drawbacks of the drain tubes described in both references for which solutions to overcome these drawbacks are described below. For example, problems arise in the prior art drain tubes in that the edge of the bowel is not easily and securely attached to the drain tube. A more secure and convenient attachment system for attaching an edge of the bowel to a drain tube is thus needed.

### OBJECTS AND SUMMARY OF THE INVENTION

It is an object of the present invention to provide new and improved drain tubes for intraoperative colonic irrigation apparatus and methods.

It is another object of the present invention to provide improvements to drain tubes for colonic irrigation disclosed in U.S. Pat. No. 4,637,814.

It is another object of the present invention to form a drain tube for colonic irrigation which is transparent and thereby enables the color and quality of the effluent to be clearly viewed and analyzed, e.g., for the purpose of determining when the colonic irrigation should be stopped.

Another object of the present invention is to provide a drain tube with a convenient and reliable system for securing an edge of the bowel to the drain tube.

Another object of the present invention is to provide a drain tube and irrigation apparatus which will minimize contamination of the sterile abdominal cavity and abdominal wound by organisms from the bowel.

In order to achieve these objects and others, a drain tube for colonic irrigation in accordance with the invention comprises a rigid body having a tubular portion and a side arm extending from a side of the tubular portion. The tubular portion has a proximal end adapted to be inserted into a bowel and a distal end through which an irrigation tube is inserted. The body is formed to be rigid and transparent via an injection molding process. The transparency of the body enables the effluent through the side arm to be visualized so that the colonic irrigation can be stopped when the effluent is clear.

To facilitate attachment of the edge of the bowel to the body of the drain tube, a circumferential sewing ring is arranged proximate the distal portion of the body. The sewing ring is angled rearward and includes an undercut rear surface defining a notch such that a tip of the sewing ring extends rearward of an inward end of the notch. A surgical clamp can therefore be inserted in the notch to clamp the edge of the bowel to the sewing ring and sutures can be formed through the sewing ring. The sewing ring may be made of a soft, elastic, pliable plastic separate from the body or be part of an unitary attachment device for attaching an edge of the bowel to the body. The attachment device may also include a tie seat for supporting a tie used to tie the bowel around the body.

The side arm is adapted to mate with an outflow tube at a distal end. More specifically, the distal end of the side arm has a tapered edge and includes a lip projecting outward from an inward end of the tapered edge to prevent disjoining of the outflow tube from the side arm. The distal end of the side arm also preferably has a smaller diameter than a remaining portion of the side arm such that the distal end is positionable directly within the outflow tube.

A rolled bowel sheath may be provided on the body to cover a sutured edge of the bowel. The rolled bowel sheath is retained by a ridge extending outward from an outer surface of the tubular portion. Once the edge of the bowel is sutured to the sewing ring, the bowel sheath can be deployed by unrolling it over the edge of the bowel. Sutures or ties are then used to secure the bowel sheath over the edge of bowel and the bowel onto the drain tube. This prevents fecal debris on the bowel edge from gaining access to the sterile operative field.

To facilitate insertion and retention of the bowel to the body of the drain tube, the proximal end of the body includes one or more circumferential triangular lips, each having a proximal surface angled forward to facilitate insertion of the drain tube into the bowel and a distal surface substantially perpendicular to an outer circumferential surface of the body to prevent the bowel from slipping off of the body when the bowel is secured around the drain tube with suture, tape or ties.

To construct the drain tube described above, a rigid body is formed having a tubular portion and a side arm extending from a side of the tubular portion using an injection molding process. The material to form the body is selected such that the body formed using the injection molding process is transparent and thereby enables effluent flowing through the side arm to be visualized. With the injection molding process, an inner surface of the body can be provided with a lower coefficient of friction in comparison to an inner surface of a body of a drain tube formed using, for example, a dip-molding process. The body can also be molded with threads at the distal end of the tubular portion of the body to mate with a screw cap or seal (or a closure member of an irrigating tube cover discussed below), the circumferential triangular lips at the proximal end of the body discussed above, the circumferential ridge discussed above and the integral sewing ring discussed above.

To prevent leakage of fluid from side holes of an irrigating tube upon removal of the irrigating tube from the drain tube, a cover can be provided including a sheath having a crimpable portion extending between a distal end and a proximal end with the distal end being adapted to be fixed to a distal end of the irrigating tube. The sheath defines an interior through which the irrigating tube passes. The cover also includes an end assembly or end piece coupled to the proximal end of the sheath and a coupling mechanism for coupling the end assembly to the distal end of a drain tube. An elastomeric seal may be arranged in connection with, and possibly fixed to, the end assembly. This seal prevents fluid from escaping from the sheath by passing between the outer wall of the irrigating tube and the inner wall of the end assembly. The irrigating tube slides through this seal as the sheath is deployed.

Improvements to methods for performing colonic irrigation are also disclosed, including the use of the novel drain tubes discussed above. One particular innovative technique involves the attachment of the bowel to the drain tube in which a sewing ring is formed on a body of the drain tube, the body is inserted into an open end of the bowel, an edge of the bowel is clamped to the sewing ring and then sutured to the sewing ring. The sewing ring may be formed integral with or separate from the body of the drain tube. To improve the sealing of the edge of the bowel to the sewing ring, a rolled bowel sheath is arranged on the body of the drain tube rearward of the sewing ring and after the edge of the bowel is sutured to the sewing ring, the bowel sheath is unrolled over the sutured edge of the bowel and then secured around the bowel forward of the sutured edge of the bowel. The sutured edge is thus covered by the bowel sheath.

### BRIEF DESCRIPTION OF THE DRAWINGS

The invention, together with further objects and advantages thereof, may best be understood by reference to the following description taken in conjunction with the accompanying drawings wherein like reference numerals identify the same or similar elements.

FIG. 1 is a cross-sectional view of a first embodiment of a drain tube in accordance with the invention shown coupled to an outflow tube.

FIG. 2 is a perspective view of a first embodiment of a device for attaching the drain tube in accordance with the invention to an edge of a bowel for use in a method for irrigating a colon in accordance with the invention.

FIG. 2A shows the attachment device of FIG. 2 wherein the bowel sheath is unrolled.

FIG. 3 is a view of the drain tube of FIG. 1 showing the operative use of surgical clamps to hold the edge of the bowel to the sewing ring in a method in accordance with the invention.

FIG. 4 is a view of the drain tube of FIG. 1 showing the suturing of the bowel to the sewing ring in a method in accordance with the invention.

FIG. 5 is a cross-sectional view of the drain tube shown in FIG. 1 at a preparatory stage of the colonic irrigation procedure.

FIG. 6 is a cross-sectional view of a second embodiment of a drain tube in accordance with the invention showing a unitary sewing cuff and tie seat mounted over a separately formed bowel sheath.

FIG. 6A is a perspective view of the unitary sewing ring and tie seat attachment device shown in FIG. 5.

FIG. 6B is a cross-sectional view of the unitary sewing ring and tie seat attachment device shown in FIG. 5.

FIG. 7 is a cross-sectional view of a third embodiment of a drain tube in accordance with the invention.

FIG. 8 is a cross-sectional view of an irrigating tube sheath and an irrigating tube in accordance with the invention.

### DETAILED DESCRIPTION OF THE INVENTION

Described below are several improvements to a drain tube for use in colonic irrigation apparatus and methods, and new instruments for use in colonic irrigation methods. These improvements and instruments can be used independently of one another or in any combination with one another, to the extent possible. Thus, unless stated otherwise herein, each improvement and instrument may be used in combination with each and every other improvement and instrument. Moreover, each improvement to a drain tube may be used in other drain tubes for colonic irrigation apparatus and methods and other similar internal passage irrigation apparatus and methods. Each instrument may be used with other drain tubes for colonic irrigation apparatus and methods and other similar internal passage irrigation apparatus and methods.

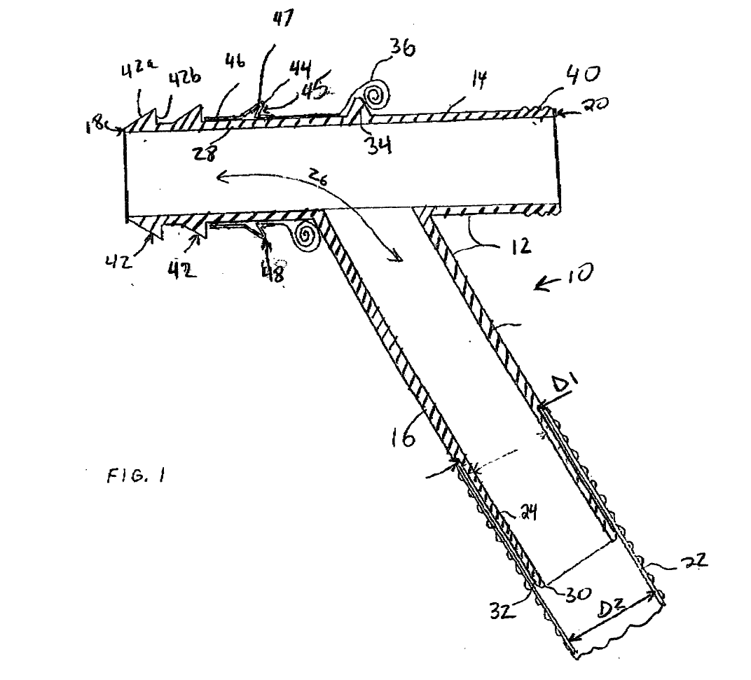

Referring first to FIG. 1, a drain tube in accordance with a first embodiment of the invention is designated generally as 10 and comprises a body 12 having a substantially tubular portion 14 and a substantially tubular side arm 16 extending from a side of the tubular portion 14. The tubular portion 14 has a proximal end 18 adapted to be inserted into the bowel of a patient and a distal end 20 adapted to operatively receive a closure and an irrigation tube. A distal end 24 of the side arm 16 is adapted to operatively connect to an outflow tube 22. A passage 26 is thus formed by a forward part 28 of the tubular portion 14 and the side arm 16 through which effluent generated during the cleaning of the colon is removed from the bowel.

The distal end 24 of the side arm 16 may be formed with various coupling structures to enable it to securely mate with the outflow tube 22. In the illustrated embodiment, the distal end 24 of the side arm 16 is formed with an outer diameter D1 to enable it to fit directly into the outflow tube 22 (which has an inner diameter D2 greater than D1). An edge 30 of the distal end 24 of the side arm 16 is optionally tapered to facilitate assembly of the outflow tube 22 around the distal end 24 of the side arm 16. To prevent the outflow tube 22 from separating or disjoining from the side arm 16, an outwardly directed lip 32 is formed at the inward end of the tapered edge 30 to project outward. Thus, while the outflow tube 22 will easily slide over the lip 32 and distal end 24 of the side arm 16 upon connection of the outflow tube 22 to the body 12, disjoining of the outflow tube 22 from the body 12 is prevented by the presence of the lip 32. Instead of the lip 32, other mechanisms can be formed during the molding of the body 12 to prevent disjoining of the outflow tube 22 from the body 12.

The body 12 of the drain tube 10 also includes a ridge 34 extending outward from an outer, circumferential surface of the tubular portion 14 of the body 12 (see FIG. 1). The ridge 34 is molded on the body 12 of the drain tube 10 to restrain a rolled bowel sheath 36 that can later be operatively deployed over the end of bowel attached to the drain tube 10 (discussed below). In the illustrated embodiment, the ridge 34 is formed at the same axial location (i.e., the same distance from the distal or proximal end) at which the side arm 16 extends from the tubular portion 14. As such, the ridge 34 does not extend completely around the tubular portion 14. A portion of the bowel sheath 36 therefore abuts against the side arm 16 and is not retained by the ridge 34. However, it is conceivable that the ridge 34 extends completely around the outer circumference of the tubular portion 14 when the ridge 34 is not formed at the same axial location from which the side arm 16 extends from the tubular portion 14.

The bowel sheath 36 may be made of molded latex or silicone, or other such elastomer, or extruded from plastic.

An important feature of the invention is that the body 12 is formed using a molding process which provides a substantially rigid plastic form, for example, an injection molding process. In contrast to the rigid plastic form obtained for the body 12 using an injection molding process, the prior art drain tubes described above are made using a dip molding process or an extruding process which provide soft, pliable forms.

A principal advantage resulting from the manufacture of the body 12 of the drain tube 10 using an injection molding process is the ability to make the body 12 transparent so that effluent flowing in passage 26 can be visualized through the body 12. Visualization of the color and quality of the effluent enables the irrigation procedure to be stopped as soon as the colon is clean. By contrast, dip-molded materials as used in conventional drain tubes cannot be made sufficiently clear to allow for an accurate assessment of the color and quality of the effluent and therefore the irrigation procedure often lasts shorter or longer than required.

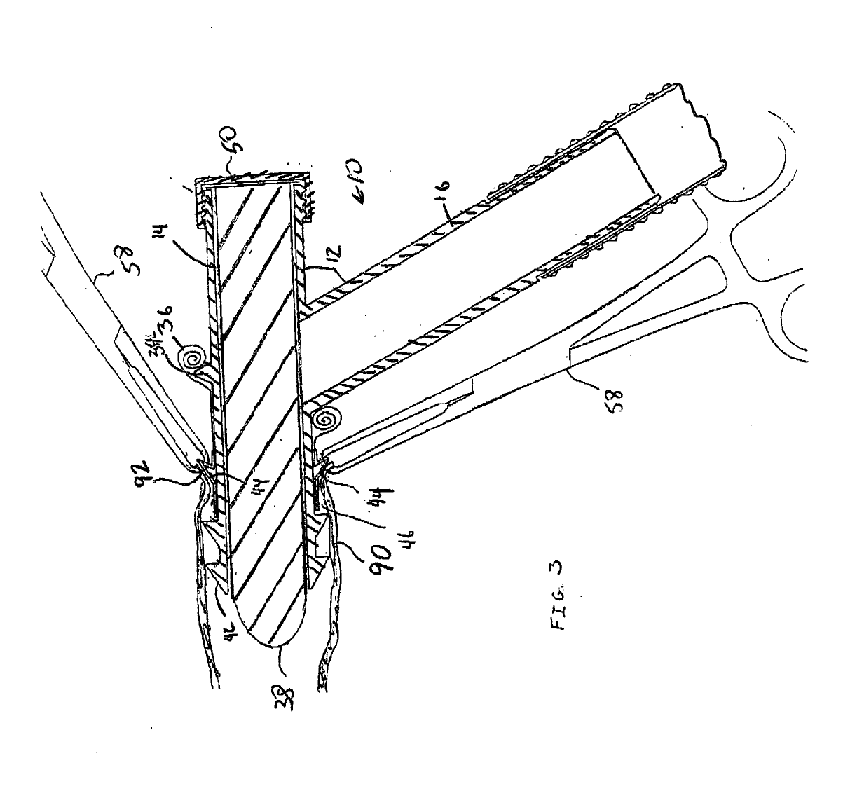

Another advantage of the manufacture of the body 12 of the drain tube 10 using an injection molding process is that the inner surface of a rigid plastic body formed using an injection molding process will have a lower coefficient of friction than that of a conventional dip-molded drain tube. A lower coefficient of friction for the inner surface of the tubular portion 14 of the body 12 permits an obturator 38 (see FIG. 3) molded of a similar rigid plastic, to slide in and out with comparative ease.

Still another advantage resulting from the manufacture of a body 12 is the ability to mold the distal end 20 of the tubular portion 14 of the body 12 with one or more threads 40 on the outer surface to provide a mechanism for attaching screw caps and seals, such as that for the irrigating tube sheath, as described below. The manner in which threads can be formed on a rigid plastic form during an injection molding process is known to those skilled in the plastic manufacturing art.

The body 12 of the drain tube 10 can also be molded via the injection molding process to include one or more circumferential lips 42 at the proximal end 18. Each lip 42 has a proximal surface 42a angled forward to facilitate insertion of the drain tube 10 into the bowel, and a distal surface 42b substantially perpendicular to the outer circumferential surface of the drain tube 10 to prevent a bowel secured to the drain tube 10 from slipping off the drain tube 10. The lips 42 therefore preferably have a substantially triangular cross-section as shown in FIG. 1. Lips 42 with the triangular cross-section can be formed via an injection molding process but cannot be formed via a dip molding process, as is used to fabricate conventional drain tubes such as those described in the above-mentioned prior art.

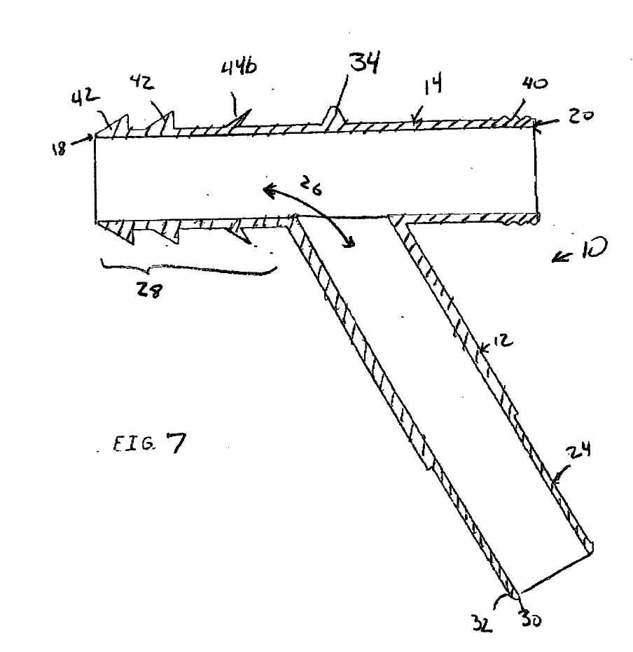

The drain tube 10 also includes a circumferential sewing cuff or sewing ring 44 arranged rearward of the lips 42 in a proximal region of the tubular portion 14 of the body 12. The sewing ring 44 helps secure the drain tube 10 within the open end of a severed bowel. The sewing ring 44 may be made of a soft, elastic, pliable plastic separate from the body 12 of the drain tube 10 (as shown in FIGS. 1,2,2A, 6, 6A and 6B). This construction is particularly applicable when the body 12 is made of a rigid plastic material. Alternatively, the sewing ring 44 may be made integral with the body 12 of the drain tube 10 (discussed below with reference to FIG. 7). This construction is particularly applicable in the event the body is made of a softer, more pliable plastic material.

The sewing ring 44 has a unique cross-sectional shape which enables the sewing ring 44 and the edge of the bowel to be grasped together easily and securely using surgical clamps. Specifically, the sewing ring 44 is angled rearward on the tubular portion 14 and includes an undercut rear surface defining a notch 45. In view of the formation of the notch 45, a tip 47 of the sewing ring 44 extends rearward of an inward end of the notch 45 thereby enabling one prong of a surgical clamp 58 to be inserted under the sewing ring 44. When the surgical clamp 58 is closed, the bowel wall is clamped to the sewing ring 44.

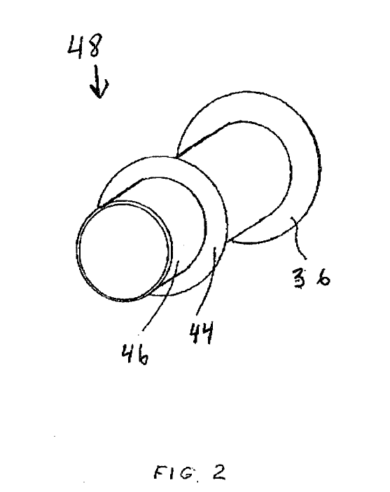

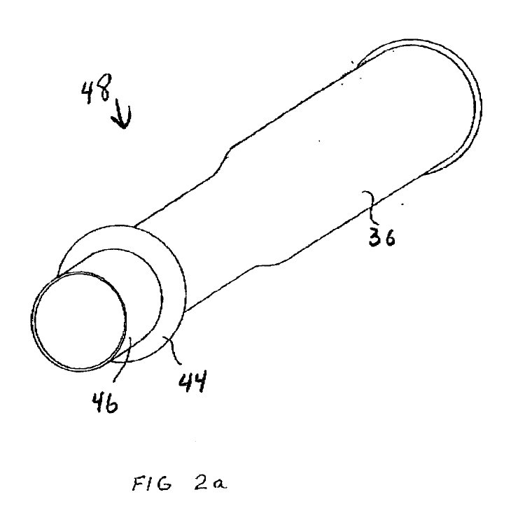

When formed separate from the body 12, the sewing ring 44 may be integral with a bowel sheath 36 and a tie seat 46. The combination of the sewing ring, bowel sheath 36 and tie seat 46 constitutes a device 48 for attaching a bowel edge to the drain tube 10 (one embodiment of such a device being shown in FIGS. 2 and 2a). When formed as an integral unit, the attachment device 48 may be formed by a dip molding process or other plastic fabrication process to have a soft, pliable construction.

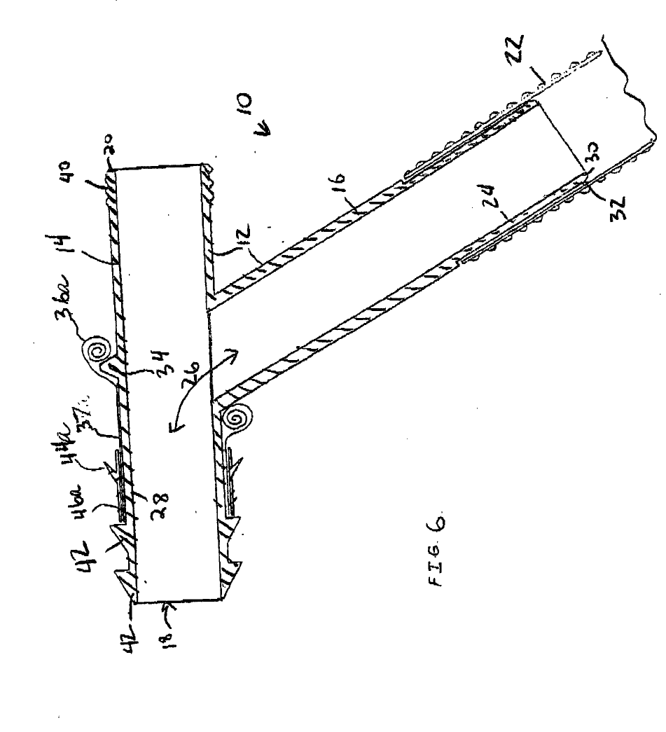

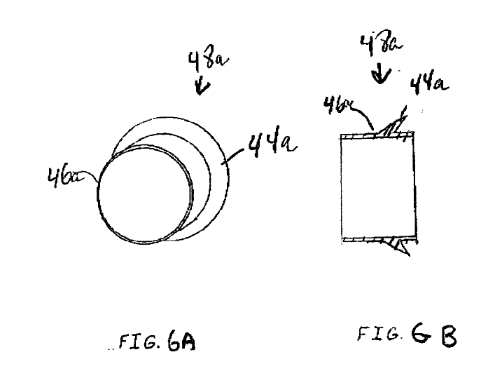

The sewing ring 44 can have various forms within the scope of the invention. As shown in FIGS. 1, 2 and 2A, the sewing ring 44 is formed by a thin membrane which is dip molded in the configuration where it is bent back on itself. This allows the tie seat 46, sewing ring 44 and bowel sheath 36 to be molded as a unit, i.e., as attachment device 48. However, an attachment device is also possible which is injection molded, and mounted over a dip molded or extruded bowel sheath. This embodiment is discussed below with reference to FIGS. 6, 6A and 6B.

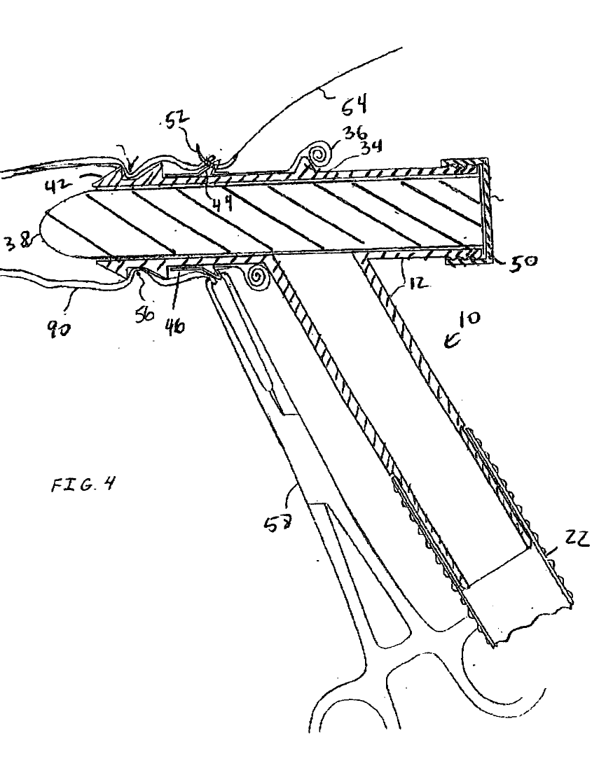

The sewing ring 44 is used to facilitate a secure coupling of the bowel wall 90 to the drain tube 10. Specifically, after the drain tube 10 is inserted into the open end of the bowel 90, the edge 92 of the open end of bowel 90 is clamped to the sewing ring 44 by clamps 58 (with one prong of the clamp 58 extending into the notch 45) and then sutured to the sewing ring 44 (see FIGS. 3 and 4). Interrupted sutures can be used at points around the circumference of the bowel 90, or a continuous circumferential suture may be used. A tie (suture thread, cloth tape, cable tie, or circular clamp) is then tied around the bowel and the drain tube 10 on the tie seat 46 between the triangular lips 42 and sewing ring 44. By suturing the bowel edge 92 to the sewing ring 44, a more secure attachment of the bowel 90 to the drain tube 10 is obtained whereby the drain tube 10 cannot disjoin from the bowel 90. If the drain tube 10 were to slip out of the bowel 90, the sterile operative field could become contaminated, increasing the likelihood of postoperative infection.

Another advantage of the placement of a sewing ring 44 on the body 12 is that it allows for rapid and secure fixation of the bowel edge 92 to the drain tube 10 with surgical clamps 58 when the drain tube 10 is first introduced into the bowel 90. This allows the surgeon to hold the inserted drain tube 10 in the bowel 90 securely, while sewing the bowel edge 92 to the sewing ring 44 and tying the bowel 90 around the drain tube 10.

In combination, the lip(s) 42 and sewing ring 44 provide a highly secure attachment of bowel 90 to the drain tube 10. Indeed, in view of the presence of the triangular lip(s) 42 and sewing ring 44, it is possible to securely couple a small diameter drain tube to bowel of large caliber so that a single drain tube 10 can be reliably secured to variously sized bowels and used to irrigate the same. By contrast, in prior art drain tubes which lack the triangular lip(s) and a sewing ring, it was often necessary to provide at least two different sizes of drain tubes, one for bowels of small caliber and another for bowels of large caliber. The attachment of large diameter bowel to a small diameter drain tube requires that the bowel be crimped or pleated around the drain tube as it is bound by a tie. When the bound bowel is crimped or pleated, it can slide more easily over the lower profile rounded lips of prior art drain tubes, making the attachment of large bowel to small prior art drain tubes insecure.

The tie seat 46 provides an annular seat which helps a surgeon position ties or clamps which bind the bowel 90 around the body 12 of the drain tube 10. In particular when the body 12 of the drain tube 10 is fabricated from a rigid plastic, it is beneficial to have an annular tie seat 46 made of an elastic material. The elastic tie seat 46 helps the surgeon form a tie that securely seals the bowel 90 around the rigid body 12 of the drain tube 10.

In one exemplifying use for colonic irrigation of a surgically opened bowel, the body 12 of the drain tube 10 is prepared with an integral attachment device 48 whereby the rolled sheath 36 is restrained by the ridge 34 (see FIG. 1). An obturator 38 is inserted into the drain tube 10 and held in place by closure 50 which is screwed to the threads 40. The outflow tube 22 is attached to the side arm 16 of the drain tube 10. In this manner, the drain tube assembly is packaged and presented to the surgeon. In surgery, the outflow tube 22 is connected at its free end to a receptacle which will receive and contain the colonic effluent (not shown). The obturator 38 facilitates introduction of the drain tube 10 into the open end of bowel 90 so that the bowel wall passes over the triangular lips 42 to the area of the sewing ring 44. The bowel wall is then clamped to the sewing ring 44 using surgical clamps 58 (see FIG. 3). Such clamping is facilitated by the presence of the notch 45 in the rear surface of the sewing ring 44. While surgical clamps 58 hold the edge 92 of the bowel 90 to the drain tube 10, the edge 92 of the bowel wall is then sutured to the sewing ring 44 with a suture needle 52 and suture thread 54 (see FIG. 4). Such suturing is also facilitated by the presence of the notch 45 in the rear surface of the sewing ring 44. The suture thread 54 is subsequently tied to secure the edge of bowel to the sewing ring 44. A tie 56 binds the bowel 90 to the drain tube 10, between the two triangular lips 42 as shown, and the surgical clamps 58 are removed.

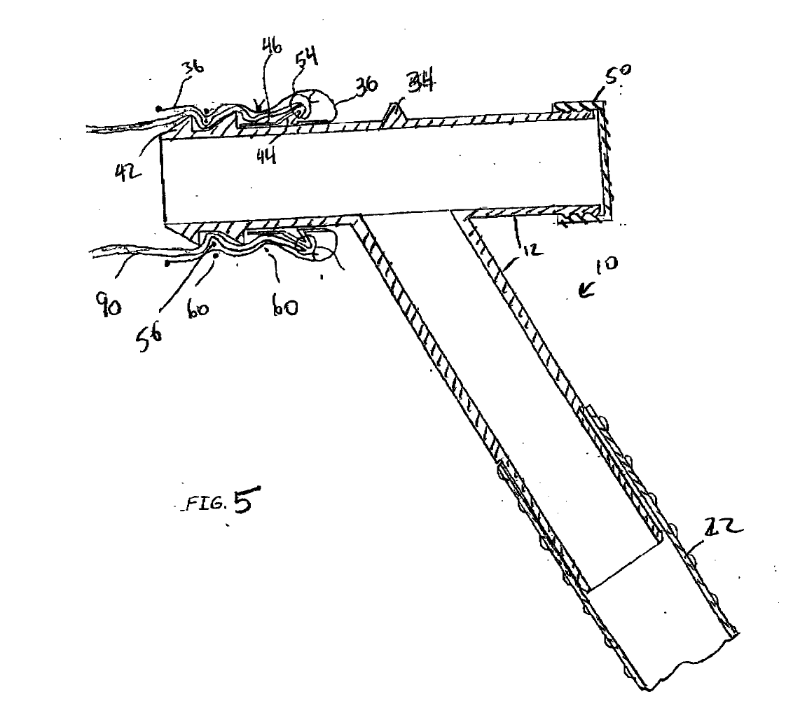

At this stage, the ridge 34 restrains the rolled bowel sheath 36 in position until it is deployed, so that it does not interfere with the actions of clamping and suturing the edge 92 of the bowel 90 to the sewing ring 44. After the edge 92 of the bowel 90 is sutured to the sewing ring 44, the sheath 36 is unrolled and deployed in a forward direction over the end of bowel (see FIG. 5). Additional ties 60 are placed around the sheath 36, bowel and proximal end of the tubular portion 14, for example, one between the two triangular lips 42 as shown and another on the tie seat 46 of the attachment device 48. Once tied around the bowel, the sheath 36 shields the sterile operative field from the contaminated end of bowel. An attachment mechanism for attaching the drain tube 10 to the bowel 90 might thus be considered to include the attachment device 48 along with the triangular lips 42 and ties 56,60.

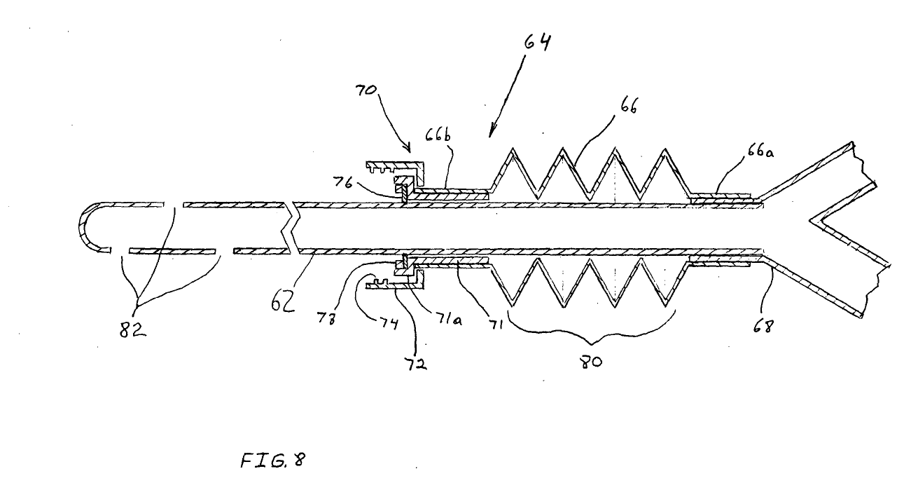

The closure member 50 is then unscrewed from the body 12 and the obturator 38 removed from the tubular portion 14. An irrigating tube 62 (shown in FIG. 8) is then passed through the tubular portion 14 and sealed at the distal end of the tubular portion 14. Fluid is directed through the irrigating tube and through perforations 82 in the irrigating tube into the bowel. Effluent generated during the cleaning process passes out of the bowel through the passage 26 into the side arm 18 and into the outflow tube 22. When the effluent appears clear in the side arm 16, as can be easily determined in view of the transparent nature of the body 12, no further fluid is infused and suction may be applied to the irrigating tube 62 to aspirate any remaining fluid in the bowel. The bowel has now been cleansed.

Referring now to FIGS. 6, 6A and 6B, a second embodiment of a drain tube in accordance with the invention includes alternate embodiments of the sewing cuff, tie seat and bowel sheath. In this embodiment, in the attachment device 48a, the sewing ring 44a is integral with the tie seat 46a, but the bowel sheath 36a is separate. The attachment device 48a can be injection molded and mounted over a dip molded or extruded bowel sheath 36.

In the manufacture of the body 12, the integral sewing ring 44a and tie seat 46a may be placed over an unrolled portion 37 of the sheath 36a and around the tubular portion 14. As such, the attachment device 48a (comprised of the integral sewing ring 44a and tie seat 46a) secures the bowel sheath 36a to the tubular portion 14.

It is also conceivable that the sewing ring is formed separate from the tie seat. In this case, it is possible that the bowel sheath, the sewing ring and the tie seat are formed each as a separate component and assembled individually around the body 12 of the drain tube 10. The bowel sheath may be separately secured around the tubular portion 14 by adhesive or solvent, or held in place only by a sewing ring

Referring now to FIG. 7, another embodiment of a drain tube 10 in accordance with the invention has a body 12 with an integrated sewing ring 44b. That is, the sewing ring 44b is molded as an integral part of the body 12 of the drain tube 10, in which case, the body 12 would preferably be injection-molded from a non-rigid elastomer.

Referring now to FIG. 8, an irrigating tube 62 used in conjunction with any of the embodiments of the drain tube 10 may be used with an irrigating tube cover 64 which prevents exposure of the contaminated irrigating tube to the sterile operative field and leakage of fluid from the irrigating tube 62 into the operative field, in particular when the irrigating tube is removed from the bowel. The irrigating tube cover 64 includes an irrigating tube sheath 66 and end assembly 70 which is specially designed to mate with the threads 40 at the distal end 20 of the body 12 of the drain tube 10. The irrigating tube sheath 66 may be formed from extruded plastic.

The irrigating tube sheath 66 has a crimped disposition as shown in FIG. 8 and the distal end 66a of the sheath 66 is fixed to a distal end or a hub 68 of the irrigating tube 62. The irrigating tube cover 64 includes an end assembly 70 which includes fitting 71 which is fixed to a proximal end 66b of the sheath 66 and which is attachable to the threads 40 at the distal end 20 of the body 12 of the drain tube 10. This attachment may be achieved using a closure member 72 having inwardly facing threads 74, and which may be coupled to and rotate around fitting 71. Alternative attachment devices to effect a secure attachment of the end assembly 70 to the distal end 20 of the body 12 of the drain tube 10 can be utilized in the invention. For example, a bayonet type closure may be utilized.

The end assembly 70 may house a circular elastomeric seal 76, in the expanded forward portion 71 a of fitting 71. The seal 76 helps seal the opening between the wall of the drain tube 10 and the wall of the irrigating tube 62 when the closure member 72 is engaged and in contact with the drain tube 10. This prevents fluid from escaping from the distal end 20 of the body 12 of the drain tube 10 when the irrigating tube 62 is in place. The irrigating tube 62 can be easily passed forward and backward through the seal 76. The seal 76 may be fixed to the end assembly 70 by an appropriate fixing mechanism, such as a grommet 78 as shown in FIG. 8. Although the end assembly 70 is shown as a combination of multiple parts, it is conceivable that it may be formed as an integral part.

For use, the irrigating tube cover 64 is initially fixed to the irrigating tube 62 (at their distal ends) and then the irrigating tube 62 is passed through the tubular portion 14 of the drain tube 10 to be positioned in the bowel. Once the irrigating tube 62 is positioned in the bowel, the end assembly 70 is attached to the distal end 20 of the body 12 of the drain tube 10 by means of the closure member 72. The bowel is then lavaged. The seal 76 prevents fluid leakage from the drain tube 10 around the irrigating tube 62. When the irrigating tube 62 is withdrawn from the bowel and drain tube 10, the irrigating tube cover 64 spreads (by expansion of the crimped portion 80 of the sheath 66) to envelope the irrigating tube 62. This prevents the sterile operative field from being exposed to the irrigating tube 62 which has been contaminated with bowel effluent by virtue of having been situated within the bowel during the cleaning process.

The irrigating tube cover 64 is particularly useful when a segment of an irrigating tube 62 contains multiple perforations 82. The cover 64 then protects the sterile field from contaminated fluid which may leak out through the perforations 82 when the irrigating tube 62 is withdrawn from the bowel.

The irrigating tube cover 64 may be used to cover various irrigating tubes which are designed to pass through drain tubes having a threaded distal end.

While particular embodiments of the invention have been shown and described, it will be obvious to those skilled in the art that changes and modifications may be made without departing from the invention in its broader aspects, and, therefore, the aim in the appended claims is to cover all such changes and modifications as fall within the true spirit and scope of the invention.

1. A drain tube for colonic irrigation, comprising
a rigid body having a tubular portion and a side arm extending from a side of said tubular portion, said tubular portion having a proximal end adapted to be inserted into a bowel and a distal end through which an irrigation tube is inserted, said side arm being adapted to mate with an outflow tube at a distal end.

2. The drain tube of claim 1, wherein said distal end of said side arm has a tapered edge.

3. The drain tube of claim 2, wherein said distal end of said side arm includes a lip projecting outward from an inward end of said tapered edge to prevent disjoining of the outflow tube from said side arm.

4. The drain tube of claim 1, wherein said distal end of said side arm has a smaller outer diameter than a remaining portion of said side arm such that said distal end is positionable directly within the outflow tube.

5. The drain tube of claim 1, further comprising a rolled bowel sheath.

6. The drain tube of claim 5, wherein said body includes a ridge extending outward from an outer surface of said tubular portion, said ridge being arranged to retain said bowel sheath.

7. The drain tube of claim 6, wherein said ridge is circumferential, extending outward from an outer, circumferential surface of said tubular portion.

8. The drain tube of claim 5, wherein said bowel sheath is made of molded latex or silicone.

9. The drain tube of claim 5, further comprising securing means for securing an unrolled portion of said bowel sheath to said body.

10. The drain tube of claim 1, wherein said body is formed from an injection molding process.

11. The drain tube of claim 1, wherein said body is transparent.

12. The drain tube of claim 1, wherein said distal end portion of said tubular portion of said body includes at least one thread for mating with a screw cap or seal.

13. The drain tube of claim 1, wherein said body includes at least one circumferential triangular lip arranged at said proximal end of said tubular portion.

14. The drain tube of claim 13, wherein each of said at least one triangular lip has a proximal surface angled forward to facilitate insertion of said drain tube into the bowel.

15. The drain tube of claim 14, wherein each of said at least one triangular lip further includes a distal surface substantially perpendicular to an outer circumferential surface of said body to prevent the secured bowel from slipping off of said body.

16. The drain tube of claim 13, further comprising a circumferential sewing ring arranged rearward of said at least one triangular lip for securing said body within an open end of the severed bowel.

17. The drain tube of claim 1, further comprising a circumferential sewing ring arranged proximate said distal portion of said body for securing said body within an open end of the severed bowel.

18. The drain tube of claim 17, wherein said sewing ring is angled rearward and includes an undercut rear surface defining a notch such that a tip of said sewing ring extends rearward of an inward end of said notch.

19. The drain tube of claim 17, wherein said sewing ring is part of an unitary attachment device for attaching an edge of the bowel to said body, said attachment device further comprising a tie seat for supporting a tie used to tie the bowel around said body.

20. The drain tube of claim 19, wherein said unitary attachment device further comprises a rolled bowel sheath adapted to roll over and cover the area of attachment of said bowel to said body.

21. The drain tube of claim 19, wherein said body includes a ridge extending outward from an outer surface of said tubular portion, further comprising a rolled bowel sheath separate from said unitary attachment device, said ridge being arranged to retain said bowel sheath, said unitary attachment device being arranged over an unrolled portion of said bowel sheath to thereby secure said bowel sheath to said body.

22. The drain tube of claim 17, wherein said sewing ring is made of a soft, elastic, pliable plastic and is separate from said body.

23. A cover for an irrigating tube, comprising:
a sheath having a crimpable portion extending between a distal end and a proximal end, said distal end being adapted to be fixed to a distal end of the irrigating tube, said sheath defining an interior through which the irrigating tube passes;
an end assembly coupled to said proximal end of said sheath; and
coupling means for coupling said end assembly to an end of a drain tube.

24. The cover of claim 23, wherein said coupling means comprise a closure member having at least one thread.

25. The cover of claim 23, further comprising an elastomeric seal arranged in connection with said end assembly for sealing the gap between the outer surface of an irrigating tube and the inner surface of said end assembly.

26. The cover of claim 25, further comprising fixing means for fixing said seal to said end assembly.

27. A method for constructing a drain tube for use in intestinal irrigation, comprising the steps of:
forming a rigid or semi-rigid body having a tubular portion and a side arm extending from a side of the tubular portion using an injection molding process; and
selecting material to form the body such that the body formed using the injection molding process is transparent and thereby enables effluent flowing through the side arm to be visualized.

28. The method of claim 27, wherein the body is formed using the injection molding process such that an inner surface of the body has a lower coefficient of friction in comparison to an inner surface of a body of a drain tube formed using a dip-molding process.

29. The method of claim 27, further comprising the step of molding a distal end of a tubular portion of the body with at least one thread on an outer surface adapted to mate with a screw cap or seal.

30. The method of claim 27, further comprising the step of molding at least one circumferential triangular lip at a proximal end of the body having a proximal surface angled forward to facilitate insertion of the drain tube into the bowel and a distal surface substantially perpendicular to an outer circumferential surface of the body to prevent a bowel secured to the body from slipping off the body.

31. The method of claim 27, further comprising the step of molding a ridge on an outer surface of a tubular portion of the body.

32. The method of claim 27, further comprising the step of molding a sewing ring on an outer, circumferential surface of a tubular portion of the body.

33. The method of claim 27, further comprising the step of arranging a unitary attachment device on the body, the unitary attachment device including a sewing ring, an elastic tie seat and a bowel sheath.

34. The method of claim 27, further comprising the step of arranging a unitary attachment device on the body, the unitary attachment device including a sewing ring and an elastic tie seat.

35. The method of claim 34, further comprising the step of injection molding the sewing ring and tie seat.

36. The method of claim 35, further comprising the step of arranging a separately formed bowel sheath on the tubular body so that at least the rolled portion of the bowel sheath sits rearward of the sewing ring on the tubular body.

37. The method of claim 36, comprising the step of mounting the injection molded sewing ring and tie seat over an unrolled portion of the bowel sheath in order to help secure the bowel sheath to the tubular body.

38. The method of claim 27, further comprising the step of forming a distal end of the side arm with a smaller diameter than a remaining portion of the side arm such that the distal end of the side arm fits directly within an outflow tube.

39. The method of claim 38, further comprising the step of providing the distal end of the side arm with a tapered edge and a lip projecting outward from an inward end of the tapered edge.

40. A drain tube for colonic irrigation, comprising
a body having a tubular portion and a side arm extending from a side of said tubular portion, said tubular portion having a proximal end adapted to be inserted into a bowel and a distal end through which an irrigation tube is inserted, said side arm being adapted to mate with an outflow tube at a distal end,
said body includes at least one circumferential triangular lip arranged at said proximal end of said tubular portion, said at least one triangular lip having planar surfaces.

41. The drain tube of claim 40, wherein each of said at least one triangular lip has a proximal surface angled forward to facilitate insertion of said drain tube into the bowel.

42. The drain tube of claim 41, wherein each of said at least one triangular lip further includes a distal surface substantially perpendicular to an outer circumferential surface of said body to prevent the secured bowel from slipping off of said body.

43. The drain tube of claim 40, further comprising a circumferential sewing ring arranged rearward of said at least one triangular lip for securing said body within an open end of the severed bowel.

44. The drain tube of claim 40, wherein said at least one triangular lip comprises a plurality of triangular lips.

45. A drain tube for colonic irrigation, comprising
a body having a tubular portion and a side arm extending from a side of said tubular portion, said tubular portion having a proximal end adapted to be inserted into a bowel and a distal end through which an irrigation tube is inserted, said side arm being adapted to mate with an outflow tube at a distal end; and
a circumferential sewing ring arranged proximate said distal portion of said body for securing said body within an open end of the severed bowel.

46. The drain tube of claim 45, wherein said sewing ring is angled rearward and includes an undercut rear surface defining a notch such that a tip of said sewing ring extends rearward of an inward end of said notch.

47. The drain tube of claim 45, wherein said sewing ring is part of an unitary attachment device for attaching an edge of the bowel to said body, said attachment device further comprising a tie seat for supporting a tie used to tie the bowel around said body.

48. The drain tube of claim 47, wherein said unitary attachment device further comprises a rolled bowel sheath adapted to roll over and cover the area of attachment of said bowel to said body.

49. The drain tube of claim 47, wherein said body includes a ridge extending outward from an outer surface of said tubular portion, further comprising a rolled bowel sheath separate from said unitary attachment device, said ridge being arranged to retain said bowel sheath, said unitary attachment device being arranged over an unrolled portion of said bowel sheath to thereby secure said bowel sheath to said body.

50. The drain tube of claim 49 wherein said ridge is circumferential, extending outward from an outer, circumferential surface of said tubular portion.

51. The drain tube of claim 45, wherein said sewing ring is made of a soft, elastic, pliable plastic and is separate from said body.

52. The drain tube of claim 45, wherein said sewing ring is integral with said body.

53. A drain tube for colonic irrigation, comprising
a body having a tubular portion and a side arm extending from a side of said tubular portion, said tubular portion having a proximal end adapted to be inserted into a bowel and a distal end through which an irrigation tube is inserted, said side arm being adapted to mate with an outflow tube at a distal end;
a rolled bowel sheath arranged on a proximal portion of said tubular portion and adapted to roll over and cover an area of attachment of the bowel to said body; and
restraining means for restraining said bowel sheath on said tubular portion

54. The drain tube of claim 53, wherein said restraining means comprise a ridge extending outward from an outer surface of said tubular portion.

55. The drain tube of claim 53, wherein said restraining means comprise a circumferential ridge extending outward from an outer, circumferential surface of said tubular portion.

56. The drain tube of claim 53, wherein said bowel sheath is made of molded latex or silicone.

57. The drain tube of claim 53, further comprising securing means for securing an unrolled portion of said bowel sheath to said body.

58. The drain tube of claim 53, wherein said bowel sheath is part of an unitary attachment device for attaching an edge of the bowel to said body, said attachment device further comprising a circumferential sewing ring arranged proximate said distal end of said body and a tie seat for supporting a tie used to tie the bowel around said body.

59. The drain tube of claim 53, further comprising a unitary attachment device for attaching an edge of the bowel to said body, said unitary attachment device being separate and apart from said bowel sheath and comprising a circumferential sewing ring arranged proximate said distal end of said body and a tie seat for supporting a tie used to tie the bowel around said body, said unitary attachment device being arranged over an unrolled portion of said bowel sheath to thereby secure said bowel sheath to said body.

60. A method for attaching a bowel to a drain tube for colonic irrigation, comprising the steps of:
forming a sewing ring on a body of the drain tube;
inserting the body into an open end of the bowel;
clamping an edge of the bowel to the sewing ring; and
suturing the edge of the bowel to the sewing ring.

61. The method of claim 60, wherein the step of forming the sewing ring on the body of the drain tube comprises the step of forming the sewing ring integral with the body of the drain tube.

62. The method of claim 60, wherein the step of forming the sewing ring on the body of the drain tube comprises the step of forming the sewing ring separate from the body of the drain tube and arranging the sewing ring around the body of the drain tube.

63. The method of claim 62, wherein the sewing ring is formed by a thin membrane which is dip molded into a configuration where it is bent back on itself, further comprising the step of molding the sewing ring, a tie seat for accommodating a tie and a bowel sheath for covering the edge of the bowel as an integral unit.

64. The method of claim 60, wherein the step of forming the sewing ring on the body of the drain tube comprises the step of forming the sewing ring with a notch in a rear surface to facilitate clamping of the edge of the bowel to the sewing ring.

65. The method of claim 60, further comprising the steps of:
arranging a rolled bowel sheath on the body of the drain tube rearward of the sewing ring;
after the edge of the bowel is sutured to the sewing ring, unrolling the bowel sheath over the sutured edge of the bowel; and then
securing the bowel sheath around the bowel forward of the sutured edge of the bowel.
